(19) United States
(12) Patent Application Publication
(10) Pub. No.: US 2009/0239972 A1
(43) Pub. Date: Sep. 24, 2009

(54) Denture Adhesive Compositions and Methods

(76) Inventors: Jayanth Rajaiah, Loveland, OH (US); Franco Silva Medeiros, Loveland, OH (US); Luisa Navarro Cerda, Cincinnati, OH (US); Robert Scott Leonard, Fairfield, OH (US); Steven Daryl Smith, Fairfield, OH (US); Mark William Hamersky, Indian Springs, OH (US); Rafael Edmundo Bras, West Chester, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/476,352

(22) Filed: Jun. 2, 2009

Related U.S. Application Data
(63) Continuation-in-part of application No. 11/590,231, filed on Oct. 31, 2006.
(60) Provisional application No. 60/735,243, filed on Nov. 9, 2005.
(60) Provisional application No. 60/760,526, filed on Jan. 20, 2006.
(60) Provisional application No. 60/735,088, filed on Nov. 9, 2005.
(60) Provisional application No. 60/760,660, filed on Jan. 20, 2006.
(60) Provisional application No. 60/735,136, filed on Nov. 9, 2005.
(60) Provisional application No. 60/760,528, filed on Jan. 20, 2006.
(60) Provisional application No. 60/735,135, filed on Nov. 9, 2005.
(60) Provisional application No. 60/760,516, filed on Jan. 20, 2006.
(60) Provisional application No. 60/734,874, filed on Nov. 9, 2005.
(60) Provisional application No. 60/760,527, filed on Jan. 20, 2006.
(60) Provisional application No. 60/760,711, filed on Jan. 20, 2006.
(60) Provisional application No. 61/058,606, filed on Jun. 4, 2008.

(51) Int. Cl. A61K 6/00

(52) U.S. Cl. 523/120

(57) ABSTRACT

The present invention relates to denture adhesive compositions. The denture adhesive compositions include an adhesive component and a viscosity index improver. The present invention is also directed to methods relating to the denture adhesive compositions.

### CROSS REFERENCE TO RELATED APPLICATION

This application is a continuation-in-part of U.S. application Ser. No. 11/590,231 filed Oct. 31, 2006 which claims priority to U.S. Provisional App. Nos. 60/735,243 filed Nov. 9, 2005; 60/760,526 filed Jan. 20, 2006; 60/735,088 filed Nov. 9, 2005; 60/760,660 filed Jan. 20, 2006; 60/735,136 filed Nov. 9, 2005; 60/760,528 filed Jan. 20, 2006; 60/735,135 filed Nov. 9, 2005; 60/760,516 filed Jan. 20, 2006; 60/734,874 filed Nov. 9, 2005 and 60/760,527 filed Jan. 20, 2006, and 60/760,711 filed Jan. 20, 2006; this application also claims priority to U.S. Provisional App. No. 61/058,606 filed Jun. 4, 2008; the substances of which are incorporated herein by reference.

### TECHNICAL FIELD

This invention relates to denture adhesive compositions and methods, in particular, to improved denture adhesive methods and compositions which include an adhesive component and a viscosity index improver.

### BACKGROUND OF THE INVENTION

Ordinary removable dentures, dental plates, partials, and the like, include teeth mounted in a suitable plate or base. While dentures are traditionally fitted for the individual user, the fit can change over time which may result in slippage or discomfort. Whether the fit is good or bad, some users prefer extra security against slippage and/or dislodgement. Denture adhesives are used to temporarily adhere the dentures to the surfaces of the oral cavity, in particular the oral mucosa and give wearers the extra security they prefer. Denture adhesives are typically applied to the denture, oral surface, or both at the beginning of the day when the dentures are placed into the oral cavity. Unfortunately, denture adhesives tend to bioerode during the course of the day due to the action of saliva, chewing, drinking, and the like. This erosion leads to loss of adhesiveness, oozing of the adhesive into the oral cavity, dislodgement, etc. As such, there is a need for improved denture adhesives.

### SUMMARY OF THE INVENTION

According to one embodiment, the present invention is directed to a denture adhesive composition, comprising a denture adhesive component and a viscosity index improver, wherein the denture adhesive composition can be dispensed from a tube.

In another embodiment, the present invention is directed to a denture adhesive composition, comprising: a) from about 10.0% to about 60.0% of a denture adhesive component comprising AVE/MA, salts of AVE/MA, mixed salts of AVE/MA, carboxymethylcellulose, or a combination thereof; b) from about 0.001% to about 20.0% of a viscosity index improver comprising microcrystalline wax, polyethylene, rubber, elastomers, or a combination thereof; and c) from about 20.0% to about 90.0% of a water insoluble component.

These and other embodiments of the present invention will be more fully understood in light of the detailed description below.

### DETAILED DESCRIPTION OF THE INVENTION

A detailed description of embodiments of the present invention is given below.

### DEFINITIONS

The abbreviation “cm”, as used herein, means centimeter. The abbreviation “mm” as used herein, means millimeter. The abbreviation “g” as used herein, means gram. The abbreviation “P” as used herein, means Pascal. The abbreviation “s” as used herein means second. The abbreviation “Ps” as used herein means Pascal—second. The abbreviation “oz” as used herein, mean ounce.

The term “denture” as used herein refers to the upper or lower denture, a partial upper or lower denture, or any combination of partial and full dentures.

The term “viscosity index improver” as used herein refers to a material which makes the viscosity and/or rheology of a material into which it is incorporated more stable as its temperature is increased over a defined range. In the case of denture adhesive products, the defined range is between about 25° C. and about 60° C.

The term “dispensed/dispensable from a tube” as used herein refers to a composition which can be dispensed from a small denture adhesive tube under manual pressure. A small denture adhesive tube is made of a foil laminate, is about 3.5 inches long, about 0.48 inches wide, and holds about 0.25 oz of product. The internal diameter of the nozzle on the small denture adhesive tube is about 0.19 inches and the nozzle length is about 0.38 inches. An example of a small denture adhesive tube is a 0.25 oz sample size tube which is supplied by Alcan Corporation as stock item number 2293.

By “safe and effective amount”, as used herein, is meant an amount of an agent high enough to significantly (positively) modify the condition to be treated or positively modify the benefit sought, but low enough to avoid serious side effects (at a reasonable benefit/risk ratio), within the scope of sound medical/dental judgment. The safe and effective amount of an agent may vary with the particular condition being treated, the age and physical condition of the patient being treated, the severity of the condition, the duration of treatment, the nature of concurrent therapy, the specific form of the source employed, and the particular vehicle from which the agent is applied.

The term “AVE/MA” as used herein refers to alkyl vinyl ether-maleic acid or anhydride copolymer. The term “mixed salts”, as used herein, refers to salts of polymers, such as AVE/MA, where at least 2 different cations are mixed on the same polymer with each other or with other salts.

The term “toxicologically-acceptable”, as used herein, is used to describe materials that are suitable in their toxicity profile for administration to humans and/or animals.

The term “non-aqueous”, as used herein, means the composition is substantially free of added water. Substantially free means that no free water is added to the composition, but the composition may contain about 5% or less of water which comes in as part of other components.

The term “water-insoluble” as used herein refers to a material that, when exposed to water, does not dissolve, but may disperse to varying degrees. Generally, a material is water-insoluble if it is less than about 10% soluble in water.

The term “bioerodible” as used herein means that the composition, when exposed to water or saliva, will erode over time due to physical and/or chemical action. The composition may erode completely or substantially, however ultimately the composition will lose its original form and/or integrity. For example, after application and use for at least about 24 hours in the oral cavity the composition will not have sufficient product integrity to easily separate or peel, in its original form, from the denture or oral surface.

Unless otherwise noted, the term “melting point” as used herein refers to the Drop Melting Point which is the temperature at which the material becomes sufficiently fluid to drop from the thermometer used in making the determination under prescribed conditions as listed in ASTM D-127. If ASTM D-127 is not suitable for the material in question, then ASTM D-3954 can be used instead.

Unless otherwise noted, the term “derivative” as used herein refers to when the primary polymeric backbone is left unchanged, but the side groups/chains and/or end groups are changed.

As used herein, the term “silicone” refers to siloxane polymers based on a structure of alternate silicon and oxygen atoms with various organic radicals attached to the silicon.

All other percentages used herein are by weight of the composition unless otherwise indicated.

All measurements referred to herein are made at 25° C. unless otherwise specified.

### Denture Adhesive Compositions and Methods

Denture adhesive compositions have become a daily product for many people who are looking for better fit and/or more security when wearing dentures. This has driven consumer demand for products which have improved properties like long lasting hold, for example. The present denture adhesive compositions deliver improvements on such desirable properties.

In general, the denture adhesive compositions of the present invention include an adhesive component and a viscosity index improver. Historically, viscosity index improver was a term associated with the lubricant industry. The viscosity of a lubricant is closely related to its ability to reduce friction. The most desirable lubricant is one which will allow the easiest movement of two surfaces while still forcing the two moving surfaces apart, because this results in the lowest friction. However, as the viscosity of liquids tends to decrease as the temperature increases, many lubricants which work at lower temperatures are not thick enough to work at higher temperatures and those that are thick enough at the higher temperatures have a tendency to be too thick to work at the lower temperatures.

For example, the automotive industry requires lubricants which can perform across a wide range of conditions, like those found in an engine. Automotive lubricants must reduce friction between engine components when it is started from cold (relative to engine operating temperatures) as well as when it is running (up to 200° C.). The best oils (i.e. lubricants) will not vary much in viscosity over such a temperature range and therefore will perform well throughout.

In order to better predict the range of temperatures at which a lubricant would work, the Society of Automotive Engineers established the Viscosity Index. The Viscosity Index highlights how a lubricant's viscosity changes with variations in temperature. The Viscosity Index shows the viscosity of materials at an arbitrary “low” temperature of 100° Fahrenheit (40° C.) and an arbitrary “high” temperature of 210° F. (100° C.).

After understanding the properties of lubricants over the set temperature ranges, it was discovered that adding certain types of compounds to the lubricants would make the viscosity of the lubricants more consistent through a broader temperature range. Thus, there was less of a decrease in the viscosity of the lubricant at the higher temperatures. Having a higher viscosity at the higher temperature allowed the lubricants to work better at the higher temperatures. The materials added to increase the viscosity at higher temperatures were defined as viscosity index improvers.

It has surprisingly been discovered that application of that principal also has relevance to denture adhesives. In general, denture adhesive compositions comprise a denture adhesive component (salts of AVE MA, for example) dispersed in a water insoluble component (petrolatum, for example). During use, the moisture in the saliva penetrates through the water insoluble component and hydrates the denture adhesive component. This makes the denture adhesive component sticky to the mucosal tissue and denture surface. The amount of hydration is influenced by, the amount of denture adhesive component, the amount of water insoluble vehicle, and the viscosity of the water insoluble vehicle, all three of which contribute to the overall viscosity of the denture adhesive composition. The viscosity of the denture adhesive composition contributes to the rate and/or amount of hydration of the denture adhesive component. Over time, excess hydration due to excess saliva and/or liquids can lead to loss of some of the adhesive, thereby weakening it. As such, a denture adhesive composition which has a higher viscosity at mouth temperature due at least in part to the water insoluble vehicle would be more resistant to hydration. Simply put, the temperature-resistance of the viscosity imparted by the viscosity index improver results in resistance to excess hydration, which in turn results in more adhesive being retained over time—leading to extended and improved performance of the denture adhesive.

Thus, the use of viscosity index improvers alone or in combination with a water insoluble component will improve the hydration characteristics of a denture adhesive and thus provide an improved hold. The temperature range most relevant for denture adhesives is from room temperature (about 25° C.) which deals with the viscosity of the denture adhesive in the dispenser (tube or package, for example) to about 40° C. which deals with the viscosity of the denture adhesive composition in the mouth. While the temperatures in the mouth can reach upward of 60° C. when drinking a hot beverage, looking at the behavior of the compositions at 40° C. tends to be a good predictor of having increased beneficial properties at 60° C. as well. Thus, viscosity index improvers relevant for denture adhesives will make the viscosity more stable over the range of functional temperatures (i.e. about 25° C. to about 60° C.).

In light of the above, in one embodiment the denture adhesive composition comprises a homogeneous mixture of the denture adhesive component and the viscosity index improver. In another embodiment, the denture adhesive composition comprises a uniform mixture of the adhesive component dispersed within the viscosity index improver.

### Denture Adhesive Component

The present invention comprises a safe and effective amount of a denture adhesive component, generally at a level from about 5% to about 60% by weight of the denture adhesive composition. In other embodiments, the denture adhesive component is in the range from about 5%, 10%, 15%, 20%, 25%, 30%, 35%, 40% to about 20%, 30%, 40%, 50%, 55%, 60%, or any combination thereof. In other embodiments, the composition of the present invention comprises at least about 20% or at least about 30% by weight of the composition of a denture adhesive component.

In general, denture adhesive components are hydrophilic particles that become sticky when activated by moisture or are hydrophilic liquids. For those that activate with moisture, moisture can be present, for example, in the denture adhesive composition itself as well as in the oral cavity of the user. In varying embodiments, the denture adhesive components herein are mucoadhesive, hydrophilic, water soluble, have the property of swelling upon exposure to moisture, form a mucilaginous mass when combined with moisture, or any combination thereof. In a further embodiment the denture adhesive component is selected from the group consisting of: glycerin, poloxamer, Sorbitol, polyox, carbomer, polyacrylamides, poly peptides, natural gums; synthetic polymeric gums; AVE/MA; AVE/MA/IB; copolymers of maleic acid or anhydride and ethylene, styrene, and/or isobutylene, polyacrylic acid and/or polyacrylates thereof; polyitaconic acid, mucoadhesive polymers; water-soluble hydrophilic colloids; saccharide; cellulose; their derivatives, and combinations thereof. Examples of such materials include karaya gum, guar gum, gelatin, algin, sodium alginate, tragacanth, chitosan, acrylamide polymers, carboxypolymethylene, polyvinyl alcohol, polyamines, polyquarternary compounds, polyvinylpyrrolidone, polyvinylpyrrolidone copolymers, cationic polyacrylamide polymers, salts and mixed salts of AVE/MA, salts and mixed salts of AVE/MA/IB, salts and mixed salts of AVE/MA/Styrene, salts and mixed salts of AVE/MA/Ethylene; polymeric acids, polymeric salts, and copolymers thereof; polyitaconic acid salts, polyhydroxy compounds, their derivatives, and combinations thereof.

In one embodiment the denture adhesive component is selected from the group consisting of salts of AVE/MA, mixed salts of AVE/MA, cellulose derivatives (such as methylcellulose, carboxymethylcellulose, hydroxyethylcellulose, hydroxypropylcellulose, hydroxy-propylmethylcellulose, corn starch, and combinations thereof), polyethylene glycol, karaya gum, sodium alginate, chitosan, and combinations thereof. In yet another embodiment, the adhesive component is selected from the group consisting of mixed salts of AVE/MA, cellulose derivatives, and combinations thereof.

In another embodiment, the denture adhesive component is selected from the group consisting of: cellulose, cellulose derivatives, starch, starch derivatives, saccharide, saccharide derivatives, polyethylene oxides, polyethylene glycols, polyvinyl alcohols, carrageenan, alginates, karaya gums, xanthan gums, guar gums, gelatins, algins, tragacanth, chitosan, acrylamide polymers, carboxypolymethylenes, polyamines, poly quaternary compounds, polyvinylpyrrolidone, AVE/MA, salts of AVE/MA, mixed salts of AVE/MA, polymeric acids, polymeric salts, polyhydroxy compounds, and combinations thereof.

In one embodiment, the adhesive component is a salt of a polymer of AVE/MA. In another embodiment the adhesive component comprises a mixed salt of a polymer of AVE/MA. In a further embodiment, the AVE/MA copolymer contains a cationic salt function comprising a cation selected from the group consisting of: Group IA and Group IIA cations of the periodic table, yttrium, titanium, zirconium, vanadium, chromium, manganese, iron, nickel, copper, zinc, boron, aluminum, and combinations thereof. In another embodiment, the adhesive component is a mixed salt of an AVE/MA copolymer containing a cationic salt function comprising a cation selected from the group consisting of strontium, zinc, iron, boron, aluminum, vanadium, chromium, manganese, nickel, copper, yttrium, titanium, magnesium, calcium, sodium, and combinations thereof. In yet another embodiment the cation is selected from the group consisting of strontium, zinc, iron, magnesium, calcium, sodium, and combinations thereof. In one embodiment, the adhesive component comprises a calcium and zinc mixed salt of an AVE/MA copolymer. In another embodiment, the denture adhesive component comprises AVE/MA, salts of AVE/MA, mixed salts of AVE MA, sodium carboxymethylcellulose, or combinations thereof.

In another embodiment, the denture adhesive component is a combination of a mixed salt of AVE/MA and carboxymethylcellulose.

In further embodiments, the denture adhesive composition comprises an additional adhesive component. In one embodiment, the additional adhesive component is present at the same levels and is selected from those listed for the adhesive component. In one embodiment, the additional adhesive component comprises a cellulose derivative. In a further embodiment, the cellulose derivative comprises sodium carboxymethylcellulose. In multiple embodiments, the additional adhesive component is present from about 5, 10, 15, 20% to about 30, 35, 40, 45, 50, 60%, or any combination thereof.

### Water Insoluble Component

In some embodiments, the present composition comprises a safe and effective amount of a water insoluble component. In one embodiment this component is present by weight of the composition at an amount from about 2, 5, 10, 20, 25, 30, 35% to about 45, 50, 60, 70, 90%, or any combination thereof. In additional embodiments the water insoluble component is present at an amount from about 20% to about 70%, from about 25% to about 60%, or from about 35% to about 60% by weight of the composition. In yet another embodiment the water insoluble component is substantially non-swellable in water. In some embodiments, the non-swellable water insoluble component swells less than about 10%, 5%, 2%, or 1% in water.

In one embodiment, the water insoluble component is selected from the group consisting of: natural wax, synthetic wax, petrolatum, polyvinyl acetate, natural oils, synthetic oils, fats, silicone, silicone derivatives, dimethicone, silicone resins, hydrocarbons, hydrocarbon derivatives, essential oils, caprilic/capric triglycerides, polybutene, oleic acid, stearic acid, and combinations thereof. In a further embodiment, the water insoluble component comprises petrolatum, polyvinyl acetate, natural oils, synthetic oils, fats, silicone, silicone derivatives, dimethicone, silicone resins, hydrocarbons, hydrocarbon derivatives, polybutene, oleic acid, stearic acid, essential oils, caprilic/capric triglycerides, or combinations thereof.

Examples of natural oils include, but are not limited to, vegetable oils (ex. corn oil), soy bean oils, cottonseed oils, palm oils, coconut oils, mineral oils, animal oils (ex. fish oils), etc. Examples of synthetic oils include, but are not limited to, silicone oils, etc. In one embodiment, the water insoluble component comprises a natural oil. In a further embodiment, the natural oil comprises mineral oil. In one embodiment, mineral oil is present in the composition at an amount from about 30% to about 50% and in another embodiment, from about 35% to about 45%. In an additional embodiment, the water insoluble component is substantially free of petrolatum. In another embodiment, the water insoluble component further comprises petrolatum.

In some embodiments, the water-insoluble component is a wax. Waxes are generally made up of various substances including hydrocarbons (normal or branched alkanes and alkenes), ketones, diketones, primary and secondary alcohols, aldehydes, sterol esters, alkanoic acids, terpenes (squalene) and monoesters (wax esters). Different types of waxes include animal and insect waxes (beeswax, Chinese wax, shellac wax, spermaceti, lanolin), vegetable waxes (bayberry wax, candelilla wax, carnauba wax, castor wax, esparto wax, Japan wax, jojoba oil, ouricury wax, rice bran wax), mineral waxes (cresin waxes, montan wax, ozocerite, peat waxes), petroleum waxes (paraffin wax), and synthetic waxes (polyethylene waxes, Fischer-Tropsch waxes, chemically modified waxes, substituted amide waxes, polymerized α-olefins).

In one embodiment the water insoluble component is a natural or synthetic wax. In a further embodiment, the natural wax is selected from the group consisting of: animal wax, vegetable wax, mineral wax, and combinations thereof. In another embodiment, the animal wax includes beeswax, lanolin, shellac wax, Chinese wax, and combinations thereof. In another embodiment, the vegetable waxes include carnauba, candelilla, bayberry, sugar cane, and combinations thereof; and mineral waxes include fossil or earth waxes (ozocerite, ceresin, montan), and petroleum waxes such as paraffin, and combinations thereof. In one embodiment the waxes herein are natural waxes selected from the group consisting of beeswax, candelilla, candela, carnauba, paraffin, and combinations thereof. In varying embodiments, wax is present in an amount from about 1, 2, 5, 8% to about 10, 20%, or any combination thereof.

### Viscosity Index Improvers

As discussed previously, viscosity index improvers make the viscosity of the denture adhesive composition more stable over a range of functional temperatures (i.e. about 25° C. to about 60° C.). It is believed that another mechanism also contributes to the improved properties of denture adhesive compositions comprising viscosity index improvers. Without being limited by theory, it is believed at least some improved properties arise when at least some of the particles of an adhesive component are at least partially coated or surrounded by a viscosity index improver. In fact, it has been surprisingly discovered that in at least some embodiments of the present invention, a viscosity index improver, microcrystalline wax for example, can at least partially coat the particles of an adhesive component. This is especially seen when the denture adhesive composition is made by heating up to or beyond the softening point of the viscosity index improver and then cooled to room temperature. In some embodiments, the viscosity index improver can coat the particles of the adhesive component by solidifying or crystallizing within the pores and/or crevices of particles of the adhesive component.

In some instances, the coating/surrounding of the adhesive component by the viscosity index improver functions as a physical barrier to protect the adhesive particles, for example, from being washed out due to incomplete hydration, excess hydration (from saliva or drinks), change in mouth temperature (ex. due to drinking a hot beverage like coffee), and/or chewing. This can also lead to a better utilization and optimization of the adhesive component which leads to a better performance. The increase in performance can lead to the ability to use less of the product to get the same or better hold as previous products.

Aside from understanding the general principal of viscosity index improvers, another way to determine whether a material would work as a viscosity index improver in a denture adhesive composition is to look at the instant viscosity ratio. The instant viscosity ratio measures the ratio of the viscosities of the prototype sample at room temperature (25° C.) and at an elevated temperature (40° C.). The present compositions tend to have a viscosity that is higher at elevated temperatures than those same compositions without a viscosity index improver. This is important because the denture adhesive composition is placed (along with the denture) into the mouth of a user which has a temperature generally higher than that of room temperature. Additionally, the temperature of a user's mouth can also be increased when ingesting hot beverages. The ability to maintain a higher viscosity at these higher temperatures contributes to better hold and less loss of the denture adhesive composition during use.

The instant viscosity ratio can be measured as outlined further below. In one embodiment, the instant viscosity ratio is greater than about 0.25. In another embodiment, the instant viscosity ratio is from about 0.25 to about 1.0. In additional embodiments, the instant viscosity ratio is from about 0.25, 0.3, 0.4, 0.6, 0.7 to about 0.3, 0.4, 0.5, 0.8, 1.0, or any combination thereof. In a further embodiment, the instant viscosity ratio is from about 0.3 to about 0.8. In other embodiments, the instant viscosity ratio is from about 0.3 to about 0.6 or from about 0.3 to about 0.5.

Some examples of viscosity index improvers include polymethacrylates, olefin copolymers, hydrogenated styrene-diene copolymers, styrene polyesters, rubber, polyvinylchloride, nylon, fluorocarbon, polyurethane prepolymer, polyethylene, polystyrene, polypropylene, cellulosic resins, acrylic resins, microcrystalline wax, elastomers, poly(n-butyl vinyl ether), poly(styrene-co-maleic anhydride), poly(alkyl fumarate co-vinyl acetate), alkylated polystyrene, poly(t-butyl styrene), or combination thereof.

Examples of polymethacrylates include, for example, polyacrylate-co-methacrylate, polymethacrylate-co-styrene, or combinations thereof. Examples of elastomers include, for example, hydrogenated styrene-co-butadiene, hydrogenated styrene-co-isoprene, ethylene-ethylene-propylene polymer, ethylene-propylene polymer, styrene-ethylene-ethylene-propylene-styrene polymer or combinations thereof. An example of a rubber includes hydrogenated polyisoprene. Other examples of viscosity index improvers can be found in “Chemistry and Technology of Lubricants,” Chapman and Hall (2^{nd} Ed. 1997).

In another embodiment, the viscosity index improver is polyethylene, such as A-C 1702 or A-C 6702 made by Honeywell, with a penetration value of about 98.5 and about 90.0, respectively, under ASTM D-1321. In another embodiment, the viscosity index improver is substantially free of amorphous polyethylene having a molecular weight of at least about 80,000. In an additional embodiment, when the viscosity index improver consists of a polyethylene having an average molecular weight of from about 1000 to about 21,000 then the adhesive component is substantially free of a mixed partial salt of a lower alkyl vinyl ether—maleic anhydride salt of calcium and alkali cations selected from the group consisting of sodium, potassium, and quaternary ammonium cations.

In another embodiment, the viscosity index improver comprises microcrystalline wax. In one embodiment, the microcrystalline wax is refined and/or substantially pure. In an additional embodiment, petrolatum does not contribute the microcrystalline wax. In another embodiment, the microcrystalline wax has a melting point ranging from about 50° C. to about 100° C. In further embodiments, the microcrystalline wax has a melting point ranging from about 50° C., 55° C., 60° C., 65° C., 70° C. to about 70° C., 75° C., 80° C., 85° C., 90° C., 95° C., 100° C., or any combination thereof. In one particular embodiment, the microcrystalline wax has a melting point ranging from about 75° C. to about 85° C. In another embodiment the microcrystalline wax is manufactured by Crompton, Sonneborn (Witco) and referred to and sold under the trademark Mutiwax® W-835. In one embodiment, the viscosity index improver comprises microcrystalline wax and is present at an amount from about 2% to about 10% and in another embodiment from about 5% to about 8%.

In some embodiments, viscosity index improvers are used in an amount from about 0.001% to about 20.0%. In another embodiment, the viscosity index improvers are used in an amount from about 1.0% to about 10.0%. In additional embodiments, the viscosity index improver is present from about 1%, 2, 5, 10, 15 to about 5, 10, 15, or 20%, or any combination thereof. In one embodiment, the viscosity index improver is water soluble and/or non-swellable in water.

### Miscellaneous Additives

### Plasticizing Agent

The compositions of the present invention may also optionally comprise a safe and effective amount of one or more toxicologically-acceptable plasticizers. In varying embodiments the level of the plasticizing agent ranges from about 0.01% to about 40%, from about 1% to about 10%, or from about 2% to about 5% by weight of the composition. In another embodiment the plasticizer is water insoluble.

Suitable plasticizing agents of the present invention include, but are not limited to, polyols (such as sorbitol); glycerin; propylene glycol; acetylated monoglyceride; hydrogenated starch hydrolysates; corn syrups; xylitol, glycerol monoesters with fatty acids; triacetin; diacetin; monoacetin; dimethyl phthalate; diethyl phthalate; dioctyl phthalate; diethylene glycol; triethylene glycol; tricresyl phosphate; dimethyl sebacate; ethyl glycolate; ethylphthalyl ethyl glycolate; o- and p-toluene ethyl sulfonamide; phthalic acid, glycerol triacetate, citric acid, phosphoric acid, glycol, a pentaerythritol ester of a fatty acid, stearic acid, glycerol monostearate, polyethylene glycol, butyl phthalyl butyl glycolate, dimethyl phthalate, dibutyl phthalate, triacetin, triethyl citrate, acetyl triethyl citrate, acetyl tributyl citrate, triphenyl phosphate, diethylene glycol, caprylic triglyceride, capric triglyceride, propylene glycol dicaprylate/caprate, their derivatives, or combinations thereof.

### Gellant Agents

The compositions of the present invention may also optionally comprise a safe and effective amount of one or more toxicologically-acceptable gellants. In varying embodiments, the level of the gellant agent ranges from about 0.01% to about 40%, from about 1% to about 10%, or from about 2% to about 5%, by weight of the composition.

Suitable gellant agents of the present invention include, but are not limited to, polyvinylpyrrolidone/eicosene copolymer sold under the tradename Ganex® V-220F from ISP; tricontanyl polyvinylpyrrolidone sold under the tradename Ganex® WP-660 from ISP; and polyamide gellants including Sylvaclear®, Sylvacote®, Sylvagel®, and Uniclear® all available from Arizona Chemical; or combinations thereof.

### Therapeutic Actives

The denture adhesive compositions may also comprise one or more therapeutic actives. Therapeutic actives may be present at a level of from about 1, 5, 10, 15, 20, 25, 30%, to about 3, 5, 10, 15, 20, 30, 50, 70%, or any combination thereof. Therapeutic actives include, for example, antimicrobial agents such as iodine, triclosan, peroxides, sulfonamides, bisbiguanides, or phenolics; antibiotics such as tetracycline, neomycin, kanamycin, metronidazole, cetylpyridinium chloride, domiphen bromide, or clindamycin; anti-inflammatory agents such as aspirin, acetaminophen, naproxen and its salts, ibuprofen, ketorolac, flurbiprofen, indomethacin, eugenol, or hydrocortisone; dentinal desensitizing agents such as potassium nitrate, strontium chloride or sodium fluoride; fluorides such as sodium fluoride, stannous fluoride, MFP (monofluorophosphate); anesthetic agents such as lidocaine or benzocaine; whitening agents such as peroxide; anti-fungals such as those for the treatment of candida albicans; insulin; steroids; herbal and other plant derived remedies; and baking soda. Other suitable therapeutic actives are discussed in the Physicians Desk Reference 62^{nd} Ed., 2008 and the Physicians Desk Reference for non-prescription drugs, dietary supplements, and herbs, 29^{th} Ed.

According to one embodiment, the active is selected from the group consisting of: anti-calculus, fluoride ion source, stannous ion source, whitening, antimicrobial, anti-plaque, anti-stain, anti-deposition, anti-gingivitis, anti-tartar, anti-periodontitis, anti-sensitivity, anti-cavity, anti-inflammatory, nutrients, antioxidants, anti-viral, anti-fungal, analgesic, anesthetic, H-2 antagonist, and combinations thereof.

### Flavor, Fragrance, and Sensate Actives

The compositions of the present invention may also include one or more components which provide flavor, fragrance, and/or sensate benefit (ex. warming or cooling agents). Suitable components include, for example, menthol, wintergreen oil, peppermint oil, spearmint oil, leaf alcohol, clove bud oil, anethole, methyl salicylate, eucalyptol, cassia, 1-8 menthyl acetate, sage, eugenol, parsley oil, oxanone, alpha-irisone, marjoram, lemon, orange, propenyl guaethol, cinnamon, vanillin, thymol, linalool, cinnamaldehyde glycerol acetal, their derivatives, and combinations thereof. In one embodiment, the active is an aromatic such as camphor, eucalyptus oil, and aldehyde derivatives such as benzaldehyde; or a combination thereof.

These agents may be present at a level of from about 0% to about 40%, in another embodiment from about 0.05 to about 5%, and in another embodiment from about 0.1 to about 2%, by weight of the composition.

### Other Miscellaneous Additives

Other suitable ingredients include colorants, preservatives (such as methyl and propyl parabens, for example), and rheology modifiers (such as silicon dioxide, for example). Rheology modifiers modify the Theological properties such as viscosity, elasticity, and or yield stress. The colorants, preservatives, and rheology modifiers may be present at levels of from about 0% to about 20%, by weight of the composition, in another embodiment from about 0.1%, 0.2, 1, 2, 5, to about 1, 5, 10, 20%, or any combination thereof.

Additionally, the compositions may also comprise one or more solvents. These optional solvents may be miscible with the viscosity index improver, water insoluble component, or both, and/or be capable of being dissipated in-situ. In one embodiment these solvents may be dissipated in-situ by evaporation, dissolution, dispersion, bio-absorption, or any other suitable means. In one embodiment, solvents include silicones, hydrocarbons, iso-dodecane, iso-hexadecane, iso-eicosane, polyisobutene, or combinations thereof.

### Denture Adhesive Composition

The denture adhesive composition can take many different forms. For example, the composition can be an emulsion, dispersion, slurry, gel, cream, paste, or combinations thereof. In one embodiment, the denture adhesive composition is in the form of a gel, cream, or paste. In another embodiment, the denture adhesive composition can be extruded out of a nozzle of a container like a tube, syringe, and/or pump, for example, directly onto a denture surface or a surface of the oral cavity.

The denture adhesive composition also has many properties. In one embodiment, the composition is bioerodible, non-aqueous, or a combination thereof. In some embodiments the composition of the present invention is bioerodible.

The denture adhesive composition and its components may contain any combination of elements and properties as disclosed herein.

### Method of Manufacture

Denture adhesive compositions can be manufactured by several methods. One example of a method for manufacturing includes: a) adding a viscosity index improver and/or water insoluble component to a vessel, b) heating and mixing the viscosity index improver and/or water insoluble component to at least about 55° C., and c) adding and mixing a denture adhesive component. The order of addition of the components is not believed to be critical so long as the adhesive component is present within the composition when the viscosity index improver and/or water insoluble component are substantially in liquid form. The temperature of the method will need to be adjusted based on the requirements for the viscosity index improver and/or water insoluble component being used.

### Composition Use

The present compositions are generally applied to the denture and/or oral cavity and thereafter the denture is secured to the oral cavity. In one embodiment the dentures are dried prior to application of the denture adhesive composition. In one embodiment it is not necessary to wet the composition and/or the denture prior to applying the composition to the denture and/or oral cavity in order to make the composition stick to the denture and/or oral cavity. The composition may be applied to any suitable location on the denture and/or oral cavity. In one embodiment the denture wearer generally wears the composition from about 1 hour to about 3 days, in another embodiment from about 6 hours to about 24 hours. After usage, the denture is removed from the oral cavity, and any remaining composition may be cleaned from the denture and/or oral cavity, for example, by gentle scrubbing with water and a brush.

### Test Methods

Procedure to Prepare the Reference Sample (RS) and Prototype Sample (PS)

The reference sample is considered the standard and is made using the standard water insoluble components, while the prototype sample is made using the viscosity index improver being tested.

### Materials

1. Standard Denture Adhesive Components and Excipient Powders (to prepare samples of both the RS and PS):
   i. Ca(47.5)/Zn(17.5) MVE/MA (Methyl Vinyl Ether/Maleic Acid) mixed partial salt (33%)
   ii. Sodium Carboxymethylcellulose (20%)
   iii. Colloidal Silicon Dioxide (1.14%)
2. Water Insoluble Components (WIC) and Viscosity Index Improver
   iv. To prepare a sample of the RS using standard WIC:
      Mineral Oil (Drakeol 35 from Penreco) (23.95%)+White Petrolatum (“Snow” from Penreco) (21.91%)
   OR
      To prepare a sample of the PS using the prototype viscosity index improver and WIC:
         Mineral Oil (Drakeol 35 from Penreco) (40.812%)+Prototype viscosity index improver (5.048%)

### Procedure

The Reference Sample and Prototype Sample are both prepared using the following procedure:

Connect a mixer with wall-scraper blades (Unimix from Haagen and Rinau) and hot water jacket to a water bath and a vacuum pump. Set the water bath of the hot water jacket to about 95° C. Add the WIC and/or viscosity index improver ingredients to mixer vessel. If the water insoluble component and/or viscosity index improver are not liquid at room temperature, allow them to soften before turning on the agitator. Turn on the agitator to about 60 RPM; mix the WIC and/or viscosity index improver ingredient(s) until their temperature reaches about 95° C. Add the “Standard Denture Adhesive Components and Excipient Powders” via a funnel to the mixer with the vent open. Close the vent and stop mixing. Scrape off powder clumps. Re-start mixing at about 60 RPM. Pull about 24 inches Hg vacuum and mix until the batch reaches about 90° C. Reduce bath temperature to about 60° C. and continue mixing under vacuum until the batch reaches about 65° C. Stop mixing, turn off the pump, slowly open the vent, release the vacuum, and raise the lid. Fill the sample into a suitable container, such as a foil tube of about 1.4 oz in capacity. Allow samples to equilibrate for about one week. Just prior to testing, squeeze out and discard approximately the first 2 grams from the tube(s).

Whenever possible, the RS and PS are made with the same denture adhesive components and excipient powders at the same levels and with the same manufacturing procedure. This is done to provide a standard matrix to test the differences between a variety of viscosity index improvers by keeping all other variables including the denture adhesive components and sample preparation procedure the same. Among other properties imparted by the standard denture adhesive components, they also provide a standard driving force for the saliva and moisture to penetrate through the denture adhesive composition, and also provide a standard matrix to test the effect of a variety of viscosity index improvers.

If it is necessary to accommodate any property of the Prototype viscosity index improver or viscosity index improver/water insoluble component combination that is not accommodated by the process detailed above (for example if it softens only at temperatures greater than 95° C.), the processing temperature profile can be modified as needed. Similarly, if the above blend of standard denture adhesive components is not suitable, then, just a single denture adhesive component, for example, sodium carboxymethylcellulose at 53%, can be used instead of the blend with Ca/Zn MVE/MA salt. Additionally, if the above testing formulation gives a PS which is too thick to test for the instant viscosity ratio as described below, then the sample may need to be diluted with additional water insoluble component like mineral oil.

The above process tests for viscosity index improvers at a level of about 5%. It is believed that testing the prototype viscosity index improvers at 5% will help set-up a baseline, meaning that a finding of viscosity index improver properties at a level of 5% is indicative of viscosity index improver properties at high levels. That being said, a prototype viscosity index improver which is tested at 5% and is found not to have viscosity index improver properties at that level may have them at a higher percentage and should be tested at a higher level to confirm.

The above process can also be scaled up and used for general manufacturing at the temperature appropriate for the viscosity index improver and/or water insoluble component of the denture adhesive composition.

### Instant Viscosity Ratio Test

The Instant Viscosity Ratio can be measured and calculated by the following procedure:

### Equipment:

Ares Strain-Controlled Rheometer

25 mm permanent parallel plates

### Method:

1. Load 25 mm parallel plates onto an Ares rheometer.
2. Zero the normal force.
3. Zero the gap (25° C. (i.e. room temperature).
4. Apply the sample to the bottom plate in a semi circular motion moving across the plate. There should be enough specimen such that when a gap of 2.177±0.005 mm is reached and excess is trimmed, the specimen extends evenly to all edges of the plate with no gaps present.
5. Adjust the Gap using the following procedure:
   Click on set gap icon. Set command gap position to 2.55 mm.
   Set the Max Force Allowed to 100 g.
   Click on set Gap.
   Trim sample with plastic cover slide.
   Set the command gap position to 2.177 mm, Max Force Allowed=100 g.
   Click on set Gap.
   Trim sample with plastic cover slide.
   Set command gap position to 2.147 mm. Max Force Allowed=100 g.
   Click on set Gap.
   Do Not Trim Sample.
   Final Gap should read 2.147±0.005 mm
   Allow the temperature to equilibrate to 25° C.
   Record the Gap and the Axial Force in test notes along with any observations made.
   Start Experiment
6. Start test:
   Method is a Step Rate (Transient) test that runs the following procedure:
      i. Applies a rate of 0/s for 1 s (a 1 s delay)
      ii. Applies a rate of 5/s for 5 s
   Result should be a curve of Viscosity vs. Time
7. Record the peak viscosity (aka “Instant Viscosity”) of this curve.
8. Repeat steps 1-7 for the PS at 25° C.—a minimum of three times
9. Repeat steps 1-7 for the PS at 40° C.—a minimum of three times
10. Calculate the average value of the Instant Viscosity for the PS at 25° C., and separately at 40° C.
11. Finally, calculate
   “Instant Viscosity Ratio”=(Average Instant Viscosity for the Composition at 40° C.)/(Average Instant Viscosity for the Composition at 25° C.)

### Dislodgement Force Test

The dislodgement force can be measured and the dislodgement force ratio can be calculated according to the method described in U.S. Pat. App. Pub. No. 2007/0185235 to Rajaiah et al.

The above test methods can also be employed to evaluate consumer product compositions, components, and raw and intermediary materials (e.g. snack dough). A representative, non-limiting list of product categories includes antiperspirants, baby care, colognes, commercial products (including wholesale, industrial, and commercial market analogs to consumer-oriented consumer products), cosmetics, deodorants, dish care, feminine protection, hair care, hair color, health care, household cleaners, incontinence care, laundry, oral care, paper products, personal cleansing, disposable absorbent articles, pet health and nutrition, prescription drugs, prestige fragrances, skin care, snacks and beverages including salted snacks, special fabric care, shaving and other hair growth management products. Exemplary product forms and brands are described on The Procter & Gamble Company's website www.pg.com, and the linked sites found thereon. It is to be understood that said test methods may be useful for use in evaluating or measuring consumer products, components, and raw and intermediary materials (e.g. snack dough), that are part of product categories other than those listed above are also contemplated.

Exemplary products within the laundry category include detergents bleach, conditioners, softeners, anti-static products, and refreshers (including liquid refreshers and dryer sheets). Exemplary products within the oral care category include dentifrice, mouth rinses, gum care products, tooth whitening products, and other tooth care products. Exemplary feminine protection products include pads, tampons, interlabial products, and pantiliners. Exemplary baby care products include diapers, wipes, baby bibs, baby change and bed mats, and foaming bathroom hand soap. Exemplary paper products include toilet tissues, paper towels, and facial tissues. Exemplary snack foods include fabricated snack pieces, snack pieces including a snack piece made with starches and flours and have included in it fruit and/or vegetable components, snack chips, snack crisp, potato or vegetable chips, potato or vegetable crisps, corn chips, tortilla chips, curls, puffs, potato sticks, French fries, and shoestring potatoes.

The following examples further describe and demonstrate embodiments within the scope of the present invention. The examples are given solely for the purpose of illustration and are not to be construed as limitations of the present invention. Many variations of these are possible without departing from the spirit and scope of the invention.

### EXAMPLES

The following are some non-limiting examples of certain embodiments of the present invention.

### Example I

| | | | | | E |
|---|---|---|---|---|---|
| | | | | | (Compar- |
| | A | B | C | D | ative) |
| | % | % | % | % | % |
| Ca/Zn AVE/MA Salt | 33 | 33 | 33 | 33 | 33 |
| CMC | 20 | 20 | 20 | 20 | 20 |
| Silica | 1.03 | 0.97 | 0.86 | 1.08 | 1.14 |
| Mineral Oil | 21.55 | 20.36 | 17.96 | 22.75 | 23.95 |
| Petrolatum | 19.72 | 18.62 | 16.43 | 20.81 | 21.91 |
| Saccharin and/or flavors | 0.01 | 0.01 | 0.02 | 0.01 | 0.00 |
| and/or colorants | | | | | |
| Microcrystalline Wax W-835 | 4.69 | 7.04 | 11.73 | 2.35 | 0.00 |
| (by Witco Crompton, | | | | | |
| Sonneborn) | | | | | |

### Example II

| | A | B | C | D | E |
|---|---|---|---|---|---|
| | % | % | % | % | % |
| Ca/Zn AVE/MA Salt | 33 | 33 | 33 | 33 | 33 |
| CMC | 20 | 20 | 20 | 20 | 20 |
| Silicon Dioxide | 1.03 | 0.97 | 0.86 | 1.08 | 1.14 |
| Mineral Oil | 41.18 | 37.99 | 32.41 | 43.57 | 30.86 |
| Petrolatum | 0 | 0 | 0 | 0 | 0 |
| Saccharin and/or flavors | 0.1 | 1 | 2 | 0 | 0 |
| and/or colorants | | | | | |
| Microcrystalline Wax W-835 | 4.69 | 7.04 | 11.73 | 2.35 | 15 |
| (by Witco Crompton, | | | | | |
| Sonneborn) | | | | | |

### Example III

| | A | B | C | D | E |
|---|---|---|---|---|---|
| | % | % | % | % | % |
| Ca/Zn AVE/MA Salt | 33 | 29.7 | 23.76 | 33 | 33 |
| Sodium Carboxymethyl Cellulose | 20 | 18 | 14.4 | 20 | 20 |
| Mineral Oil, Heavy, White, USP | 22.77 | 24.72 | 26.92 | 20.36 | 40.812 |
| (Kaydol) | | | | | |
| Petrolatum, White | 20.82 | 22.61 | 24.63 | 18.62 | 0 |
| Colloidal Silicon Dioxide NF | 1.08 | 0.97 | 0.97 | 0.97 | 1.14 |
| Microcrystalline Wax W835 | 2.33 | 4 | 9.32 | 7.05 | 5.048 |

### Example IV

| | A | B | C | D | E | F | G | H | I |
|---|---|---|---|---|---|---|---|---|---|
| | % | % | % | % | % | % | % | % | % |
| Ca(47.5)/Zn(17.5) | 33.00 | 31.75 | 32.38 | 33.00 | 32.38 | 31.75 | 33.00 | 31.75 | 30.51 |
| Gantrez Salt | | | | | | | | | |
| Sodium | 20.00 | 19.25 | 19.62 | 20.00 | 19.62 | 19.25 | 20.00 | 19.25 | 18.49 |
| Carboxymethyl | | | | | | | | | |
| Cellulose | | | | | | | | | |
| Mineral Oil, | 39.86 | 40.86 | 40.86 | 38.86 | 39.86 | 41.36 | 40.86 | 41.86 | 42.86 |
| Heavy, White, USP | | | | | | | | | |
| (Kaydol) | | | | | | | | | |
| Petrolatum, White | 0 | 0 | 0 | 0 | 0 | 0 | 0 | 0 | 0 |
| Colloidal Silicon | 1.14 | 1.14 | 1.14 | 1.14 | 1.14 | 1.14 | 1.14 | 1.14 | 1.14 |
| Dioxide NF | | | | | | | | | |
| Microcrystalline | 6 | 7 | 6 | 7 | 7 | 6.5 | 5 | 6 | 7 |
| Wax W835 | | | | | | | | | |

Polyethylene AC 6702 can be substituted for the microcrystalline wax in the above examples.

The above examples are made using the “Procedure to prepare the reference sample” as discussed above with all non-powder ingredients not specifically listed in the procedure being added with the water insoluble component/viscosity index improver and all powder ingredients being added with the denture adhesive component. The prototype sample in Example III-E is evaluated for instant viscosity ratio vs. the comparative example of I-E. The instant viscosity of III-E at 25° C. is 211.4 Ps and at 40° C. is 80.7 Ps. This gives an instant viscosity ratio for III-E of 0.38. In contrast to this, the comparative example of I-E, made using the combination of mineral oil and petrolatum used in traditional denture adhesive creams, has an instant viscosity at 25° C. of 289.9 Ps and at 40° C. of 51.4 Ps. This gives an instant viscosity ratio for I-E of 0.18. The higher instant viscosity ratio of Example III-E shows that it is more temperature resistant than the reference/traditional water insoluble component and thus, microcrystalline wax will work as a viscosity index improver in that denture adhesive composition.

All documents cited in the Detailed Description of the Invention are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A denture adhesive composition, comprising:
a) a denture adhesive component, and
b) a viscosity index improver,
wherein the denture adhesive composition can be dispensed from a tube.

2. The denture adhesive composition of claim 1, wherein the viscosity index improver is selected from the group consisting of polymethacrylates, olefin copolymers, hydrogenated styrene-diene copolymers, styrene polyesters, rubber, polyvinylchloride, nylon, fluorocarbon, polyurethane prepolymer, polyethylene, polystyrene, polypropylene, cellulosic resins, acrylic resins, microcrystalline wax, elastomers, poly(n-butyl vinyl ether), poly(styrene-co-maleic anhydride), poly(alkyl fumarate co-vinyl acetate), alkylated polystyrene, poly(t-butyl styrene), and combinations thereof.

3. The denture adhesive composition of claim 2, wherein the viscosity index improver is selected from the group consisting of polymethacrylates, olefin copolymers, hydrogenated styrene-diene copolymers, styrene polyesters, poly(n-butyl vinyl ether), poly(styrene-co-maleic anhydride), poly(alkyl fumarate co-vinyl acetate), alkylated polystyrene, poly(t-butyl styrene), and combinations thereof.

4. The denture adhesive composition of claim 2, wherein the viscosity index improver is selected from the group consisting of rubber, polyvinylchloride, nylon, fluorocarbon, polyurethane prepolymer, polyethylene, polystyrene, polypropylene, cellulosic resins, acrylic resins, microcrystalline wax, elastomers, and combinations thereof.

5. The denture adhesive composition of claim 2, wherein the viscosity index improver is in an amount from about 0.001% to about 20.0% by weight of the denture adhesive composition.

6. The denture adhesive composition of claim 1, wherein the viscosity index improver comprises microcrystalline wax, polyethylene, rubber, elastomers, or a combination thereof.

7. The denture adhesive composition of claim 6, wherein the viscosity index improver is in an amount from about 1.0% to about 10.0% by weight of the denture adhesive composition.

8. The denture adhesive composition of claim 5, wherein the denture adhesive component is selected from the group consisting of cellulose, cellulose derivatives, starch, starch derivatives, saccharide, saccharide derivatives, polyethylene oxides, polyethylene glycols, polyvinyl alcohols, carrageenan, alginates, karaya gum, xanthan gum, guar gum, gelatin, algin, tragacanth, chitosan, acrylamide polymers, carboxypolymethylene, polyamines, polyquaternary compounds, polyvinylpyrrolidone, AVE/MA, salts of AVE/MA, mixed salts of AVE/MA, polymeric acids, polymeric salts, polyhydroxy compounds, and combinations thereof.

9. The denture adhesive composition of claim 8, wherein the denture adhesive component is in an amount from about 10.0% to about 60.0% by weight of the denture adhesive composition.

10. The denture adhesive composition of claim 7, wherein the denture adhesive component comprises AVE/MA, salts of AVE/MA, mixed salts of AVE/MA, sodium carboxymethylcellulose, or a combination thereof.

11. The denture adhesive composition of claim 10, wherein the denture adhesive component is in an amount from about 20.0% to about 55.0% by weight of the denture adhesive composition.

12. The denture adhesive composition of claim 1, further comprising a water insoluble component.

13. The denture adhesive composition of claim 12, wherein the water insoluble component comprises petrolatum, polyvinyl acetate, natural oils, synthetic oils, fats, silicone, silicone derivatives, dimethicone, silicone resins, hydrocarbons, hydrocarbon derivatives, polybutene, oleic acid, stearic acid, essential oils, caprilic/capric triglycerides, or combinations thereof.

14. The denture adhesive composition of claim 13, wherein the water insoluble component is in an amount from about 20% to about 70% by weight of the denture adhesive composition.

15. A denture adhesive composition, comprising:
a) from about 10.0% to about 60.0% of a denture adhesive component comprising AVE/MA, salts of AVE/MA, mixed salts of AVE/MA, carboxymethylcellulose, or a combination thereof;
b) from about 0.001% to about 20.0% of a viscosity index improver comprising microcrystalline wax, polyethylene, rubber, elastomers, or a combination thereof; and
c) from about 20.0% to about 90.0% of a water insoluble component.

16. The denture adhesive composition of claim 15, wherein the water insoluble component comprises petrolatum, polyvinyl acetate, natural oils, synthetic oils, fats, silicone, silicone derivatives, dimethicone, silicone resins, hydrocarbons, hydrocarbon derivatives, polybutene, oleic acid, stearic acid, essential oils, caprilic/capric triglycerides, or combinations thereof.

17. The denture adhesive composition of claim 16, wherein the viscosity index improver comprises microcrystalline wax.

18. The denture adhesive composition of claim 17, wherein the water insoluble component comprises a natural oil comprising mineral oil.

19. The denture adhesive composition of claim 18, wherein the denture adhesive component comprises a combination of a mixed salt of AVE/MA and carboxymethylcellulose.

20. The denture adhesive composition of claim 19, wherein the water insoluble component further comprises petrolatum.
